(19) United States
(12) Patent Application Publication
(10) Pub. No.: US 2006/0056701 A1
(43) Pub. Date: Mar. 16, 2006

(54) Joint segmentation and registration of images for object detection

(76) Inventors: Gozde Unal, West Windsor, NJ (US); Gregory G. Slabaugh, Princeton, NJ (US); Jason Jenn-Kwei Tyan, Princeton, NJ (US)

Correspondence Address: Siemens Corporation;Intellectual Property Department, 170 Wood Avenue South, Iselin, NJ, 08830, US

(21) Appl. No.: 11/044,237

(22) Filed: Jan. 27, 2005

Related U.S. Application Data
(60) Provisional application No. 60/549,459, filed on Mar. 2, 2004.

(51) Int. Cl. G06K 9/48; G06K 9/32; A61B 5/00

(52) U.S. Cl. 382/199; 382/294; 600/300

(57) ABSTRACT

A method for joint segmentation and registration of a contour for detecting an object in image data includes initializing a segmentation, initializing a registration of the segmentation, updating the segmentation and the registration, outputting an updated segmentation and an updated registration, wherein the updated segmentation and updated registration delineates the object in the image data.

This application claims priority to U.S. Provisional Application Ser. No. 60/549,459, filed on Mar. 2, 2004, which is herein incorporated by reference in its entirety.

### BACKGROUND OF THE INVENTION

1. Technical Field

The present invention relates to computer aided diagnosis applications, and more particularly to a system and method for joint segmentation and registration of images for object detection, for example, lymph node detection.

2. Discussion of Related Art

Detection of specific anatomic structures in medical images or volumes is an important research problem in Computer Aided Diagnosis (CAD) applications. Detection methods include methods for segmentation for outlining a target structure and registration in the presence of multiple images of the same structure or region. In medical imaging applications multiple number of image volumes in which a structure of interest resides may be available. Different modality images of the same region may also be available in some applications. The challenge is then to make use of and relate the existing extra information from several given image volumes. Methods for dealing with the detection problem in multiple images have been proposed. In one class of methods, a target region is segmented separately in both images and the resulting boundaries or regions are registered. In another class of methods, a global or local registration of the two images is carried out and the target region is segmented using information coming from both images.

However, no known system or method exists for a combined region and boundary-based flow for coupled registration and segmentation. Therefore, a need exists for a system and method for joint segmentation and registration of images for object detection.

### SUMMARY OF THE INVENTION

According to an embodiment of the present disclosure, a method for joint segmentation and registration of a contour for detecting an object in image data comprises initializing a segmentation, initializing a registration of the segmentation, updating the segmentation and the registration, outputting an updated segmentation and an updated registration, wherein the updated segmentation and updated registration delineates the object in the image data.

The segmentation is an ellipse and the registration is rigid. Updating the segmentation and registration further comprises accumulating gradients for the segmentation evolution for a plurality of images in the image data and a plurality of samples on the ellipse, updating the registration for each of the plurality of images, and updating the segmentation given the updated registration.

The method comprises determining parameters of each rigid registration including a translation vector, a rotation matrix and a non-uniform scale matrix.

The segmentation is a surface of a three-dimensional structure and the registration is non-rigid. The non-rigid registration is a deformation vector field over the surface of the three-dimensional object. Updating the segmentation and registration further comprises accumulating gradients for surface evolution for each coordinate in the image volume, and accumulating gradients for the non-rigid registration evolution for each coordinate in the image volume. The deformation vector field maps the surface in a first image onto the surface of in a second image. The three-dimensional surface is determined from an initialized surface seed of the segmentation in a first image slice of the image data and two neighboring slices in the image data in which the surface seed is extended.

According to an embodiment of the present disclosure, a program storage device is provided readable by machine, tangibly embodying a program of instructions executable by the machine to perform method steps for joint segmentation and registration of a contour for detecting an object in image data. The method steps comprise initializing a segmentation, initializing a registration of the segmentation, updating the segmentation and the registration, and outputting an updated segmentation and an updated registration, wherein the updated segmentation and updated registration delineates the object in the image data.

According to an embodiment of the present disclosure, a method for joint segmentation and registration of a contour for detecting an object in an image volume comprises initializing a seed in a first slice of the image volume, initializing a signed distance function, converting the distance function into a three-dimensional surface seed, initializing a non-rigid registration for the surface, updating the distance function and the non-rigid registration, and outputting an updated surface and distance function mapping the surface in the first slice to the surface in a second slice, the surface delineating the object in the image volume.

Updating the segmentation and registration further comprises accumulating gradients for surface evolution for each coordinate in the image volume, and accumulating gradients for the non-rigid registration evolution for each coordinate in the image volume.

The surface is determined from an initialized surface seed of the segmentation in the first image slice of the image data and two neighboring slices in the image data in which the surface seed is extended.

### BRIEF DESCRIPTION OF THE DRAWINGS

Preferred embodiments of the present invention will be described below in more detail, with reference to the accompanying drawings:

FIG. 1 is a flow chart of a method according to an embodiment of the present disclosure;

FIG. 2 is an illustration of a system according to an embodiment of the present disclosure;

FIGS. 3A and 3B are flow charts of a method for object detection using elliptical contour with rigid registrations according to an embodiment of the present disclosure;

FIGS. 4A-4I is an illustration of a coupled registration of three synthetic ellipses using elliptical contour with rigid registrations according to an embodiment of the present disclosure;

FIG. 5 is a flow chart of a method for object detection using a general contour with non-rigid registration according to an embodiment of the present disclosure; and

FIG. 6 illustrates a transformation according to an embodiment of the present disclosure.

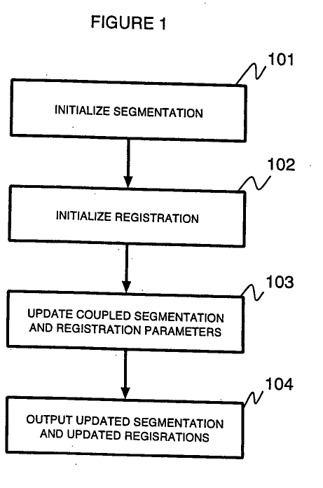

### DETAILED DESCRIPTION OF PREFERRED EMBODIMENTS

A combined region and boundary-based system and method performs coupled registration and segmentation for detecting an object an image volume, e.g., an MR or CT image volume. According to an embodiment of the present disclosure, a finite dimensional registration domain is used for object detection. The finite dimensional registration domain evolves parameters of an ellipse and its registration parameters for object detection. According to an embodiment of the present disclosure, coupled PDEs (Partial Differential Equations) are used to estimate a contour in a source image or a segmentation, and its non-rigid deformation onto a target image. The non-rigid deformation corresponds to a registration in infinite dimensions without using shape priors.

It is to be understood that the present invention may be implemented in various forms of hardware, software, firmware, special purpose processors, or a combination thereof. In one embodiment, the present invention may be implemented in software as an application program tangibly embodied on a program storage device. The application program may be uploaded to, and executed by, a machine comprising any suitable architecture.

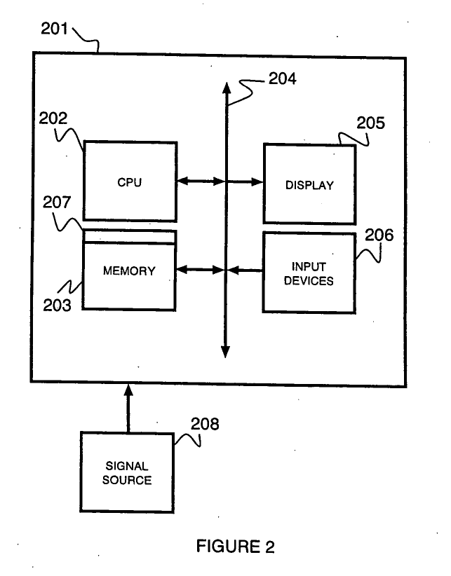

Referring to FIG. 2, according to an embodiment of the present invention, a computer system 201 for implementing a method for joint segmentation and registration can comprise, inter alia, a central processing unit (CPU) 202, a memory 203 and an input/output (I/O) interface 204. The computer system 201 is generally coupled through the I/O interface 204 to a display 205 and various input devices 206 such as a mouse and keyboard. The support circuits can include circuits such as cache, power supplies, clock circuits, and a communications bus. The memory 203 can include random access memory (RAM), read only memory (ROM), disk drive, tape drive, etc., or a combination thereof. The present invention can be implemented as a routine 207 that is stored in memory 203 and executed by the CPU 202 to process the signal from the signal source 208. As such, the computer system 201 is a general-purpose computer system that becomes a specific purpose computer system when executing the routine 207 of the present invention.

The computer platform 201 also includes an operating system and microinstruction code. The various processes and functions described herein may either be part of the microinstruction code or part of the application program (or a combination thereof), which is executed via the operating system. In addition, various other peripheral devices may be connected to the computer platform such as an additional data storage device and a printing device.

It is to be further understood that, because some of the constituent system components and method steps depicted in the accompanying figures may be implemented in software, the actual connections between the system components (or the process steps) may differ depending upon the manner in which the present invention is programmed. Given the teachings of the present invention provided herein, one of ordinary skill in the related art will be able to contemplate these and similar implementations or configurations of the present invention.

Elliptical Contour with Rigid Registrations Referring to the finite dimensional registration domain is used for object detection; given a finite number of images Iᵢ:Ωᵢ→ [symbol] ², i=1, . . . m, e.g., m=2 for illustration purposes; assume a common contour C lives on an independent domain Ω, and let C₁=g₁(C), C₂=g₂(C). Then the transformations that deform the common contour C onto contours in image domains Ωᵢ are denoted by gᵢ: [symbol] ²→ [symbol] ², each of which is an element of a finite dimensional group G with parameters wᵢ, . . . , wₙ. The goal is to find a closed curve CεΩ that deforms on the common domain whereas a curve CᵢεΩᵢ corresponding to the mapping Cᵢ=gᵢ(C) deforms on the ith image domain Ωᵢ. All contour parameters propagate according to a generic region-based energy functional defined over both image domains as follows:

$$\begin{matrix}E ( C , {g}_{1} , {g}_{2} ) = {\int }_{{C}_{1 i n}}^{} {f}^{1} ( {x}_{1} ) ⅆ {x}_{1} + {\int }_{{C}_{2 i n}}^{} {f}^{2} ( {x}_{2} ) ⅆ {x}_{2} + {\int }_{C} ⅆ s \\ = {\int }_{{C}_{ i n}}^{} {f}^{1} ( {g}_{1} ( x ) ) \vert {g}_{1}^{′} \vert ⅆ x + \\ {\int }_{{C}_{ i n}}^{} {f}^{2} ( {g}_{2} ( x ) ) \vert {g}_{2}^{′} \vert ⅆ x + {\int }_{C} ⅆ s\end{matrix} \qquad ( 1 )$$

where f₁=fᵢₙ¹−fₒᵤₜ¹, and fᵢₙ¹ and fₒᵤₜ¹ are the region descriptors inside and outside the transformed contour g₁(C) respectively (same comments apply for fᵢₙ² and fₒᵤₜ² for C₂). For example, a piecewise constant model can be utilized by choosing fᵢ=(Iᵢ−meanᵢₙ)²−(Iᵢ−meanₒᵤₜ)². A regularization on the unknown contour C is typically included as given by the last term above.

The evolution of the contour C can be given by:

$$\frac{\partial C}{\partial t} = [ {f}^{1} ( {g}_{1} ( x ) ) \vert {g}_{1}^{′} \vert + {f}^{2} ( {g}_{2} ( x ) ) \vert {g}_{2}^{′} \vert - κ ] N \qquad ( 2 )$$

where N denotes the unit normal to C, and k is the curvature of the contour. The evolution of the first registration g₁ and the second registration g₂ can be given by:

$$\begin{matrix}\frac{\partial {g}_{1}}{\partial t} = {\int }_{C} {f}^{1} ( {g}_{1} ( x ) ) 〈 \frac{\partial {g}_{1} ( x )}{\partial {w}_{i}} , {( {( {g}_{1}^{′} )}^{- 1} )}^{T} \vert {g}_{1}^{′} \vert N 〉 ⅆ s & ( 3 ) \\ \frac{\partial {g}_{2}}{\partial t} = {\int }_{C} {f}^{2} ( {g}_{2} ( x ) ) 〈 \frac{\partial {g}_{2} ( x )}{\partial {w}_{i}} , {( {( {g}_{2}^{′} )}^{- 1} )}^{T} \vert {g}_{2}^{′} \vert N 〉 ⅆ s & ( 4 )\end{matrix}$$

Referring now to an ellipse-segmenting flow with region-based energy, for specific applications in medical image analysis, the structures of interest may be approximated by an elliptical contour ε.

Incorporation of such a priori information into the evolution equation will result in reduced degrees of freedom of the evolving curve and hence lead to a more robust estimation of the contour position and its registration.

A point on a 2D ellipse parameterized by pε[0,2π) is given by

$$p ( \begin{matrix}a cos ( p ) \\ b sin ( p )\end{matrix} ) ,$$

and using its translation vector

$$t = ( \begin{matrix}d \\ e\end{matrix} ) ,$$

and rotation matrix

$$R = ( \begin{matrix}cos ( {θ}_{e} ) & sin ( {θ}_{e} ) \\ - sin ( {θ}_{e} ) & cos ( {θ}_{e} )\end{matrix} ) ,$$

the parameterization of the elliptical contour in 2D is:

$$ε ( p ) = a ( \begin{matrix}cos ( {θ}_{e} ) \\ - sin ( {θ}_{e} )\end{matrix} ) cos ( p ) + b ( \begin{matrix}sin ( {θ}_{e} ) \\ cos ( {θ}_{e} )\end{matrix} ) sin ( p ) + ( \begin{matrix}d \\ e\end{matrix} ) \qquad ( 5 )$$

Utilizing this parameterization, the variation of the energy in Eq. (1) with respect to ellipse parameters λⁱε{a,b,d,e,θₑ}, i=1, . . . , 5 yields the gradient flows:

$$\frac{ⅆ {λ}^{i}}{ⅆ t} = {\int }_{ε}^{} {f}^{1} ( {g}_{1} ( x ) ) 〈 \frac{\partial {g}_{1} ( x )}{\partial {λ}^{i}} , N 〉 \vert {g}_{1}^{′} \vert ⅆ p + {\int }_{ε}^{} {f}^{2} ( {g}_{2} ( x ) ) 〈 \frac{\partial {g}_{2} ( x )}{\partial {λ}^{i}} , N 〉 \vert {g}_{2}^{′} \vert ⅆ p \qquad ( 6 )$$

for an evolution of the ellipse with this energy. ∂_{ε}/∂λⁱ are given for each parameter by:

$$\begin{matrix}\frac{\partial ε ( p )}{\partial a} = ( \begin{matrix}cos ( {θ}_{e} ) \\ - sin ( {θ}_{e} )\end{matrix} ) cos ( p ) & ( 7 ) \\ \frac{\partial ε ( p )}{\partial b} = b ( \begin{matrix}sin ( {θ}_{e} ) \\ cos ( {θ}_{e} )\end{matrix} ) sin ( p ) & ( 8 ) \\ \frac{\partial ε ( p )}{\partial d} = ( \begin{matrix}1 \\ 0\end{matrix} ) , \frac{\partial ε ( p )}{\partial e} = ( \begin{matrix}0 \\ 1\end{matrix} ) & ( 9 ) \\ \frac{\partial ε ( p )}{\partial {θ}_{e}} = ( \begin{matrix}- a sin ( {θ}_{e} ) cos ( p ) + b cos ( {θ}_{e} ) sin ( p ) \\ - a cos ( {θ}_{e} ) cos ( p ) - b sin ( {θ}_{e} ) sin ( p )\end{matrix} ) & ( 10 )\end{matrix}$$

In Eq. (6),

$$N ( p ) = ( \begin{matrix}a sin ( {θ}_{e} ) sin ( p ) + b cos ( {θ}_{e} ) cos ( p ) \\ a cos ( {θ}_{e} ) sin ( p ) - b sin ( {θ}_{e} ) cos ( p )\end{matrix} )$$

is the normal vector of the ellipse.

For each of the rigid registrations gᵢ, a translation vector T, a rotation matrix R, and a non-uniform scale matrix S are used. Thus:

$$\frac{\partial {g}_{k}}{\partial {T}^{x}} = ( \begin{matrix}1 \\ 0\end{matrix} ) , \frac{\partial {g}_{k}}{\partial {T}^{y}} = ( \begin{matrix}0 \\ 1\end{matrix} ) , \frac{\partial {g}_{k} ( x )}{\partial θ} = ( \begin{matrix}- sin ( {θ}_{e} ) & cos ( {θ}_{e} ) \\ - cos ( {θ}_{e} ) & - sin ( {θ}_{e} )\end{matrix} )$$

$$\frac{\partial {g}_{k} ( x )}{\partial {S}_{x}} = ( \begin{matrix}1 \\ 0\end{matrix} \begin{matrix}0 \\ 0\end{matrix} ) , \frac{\partial {g}_{k} ( x )}{\partial {S}_{y}} = ( \begin{matrix}0 \\ 0\end{matrix} \begin{matrix}0 \\ 1\end{matrix} )$$

The evolution of the rigid registrations of the ellipse can be carried out by Eqs. (3-4), or it can also be incorporated into the parametric ellipse model to re-derive the evolutions of the rotation, translation and scale parameters from one image into the other. Eqs. (3-4) may be used to evolve the ellipse and the registration parameters.

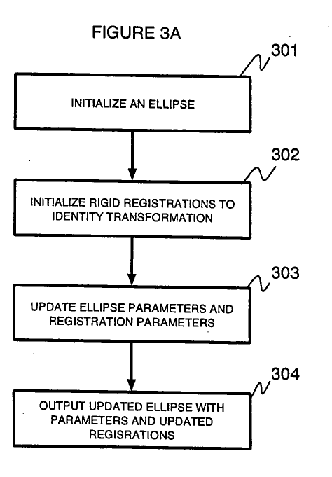

Referring to FIG. 3A, an ellipse is initialized 301, for example, by manually setting a seed position with present initial parameters for a, b, and θ. Rigid registrations are initialized 302 to the identity transformations, e.g., a translation vector T=0, a rotation matrix R=Identity, and a non-uniform scale matrix S=Identity. Ellipse parameters and registration parameters are updated 303 for a time period of interest. The ellipse is updated with parameters λⁱ and registrations are updated for gᵢ(TotalTime), yielding the ellipse on images i=1, . . . , m 304.

Referring to FIG. 3B, the following flow may be used to updating a segmentation and registration 303.

Δλⁱ(t) = 0,j = 1, . . . , n (number of parameters for ellipse)
For i = 1: m (number of images) (305)

Δgᵢ(t) = 0
For p = 0:Δp:nSamples (loop around the ellipse) (306)

x = coordinate of sample p
//Accumulate gradients, see right portion of Eq. 6 (307)
${Δλ}^{i} ( t ) += {f}^{i} ( {g}_{i} ( x ) ) 〈 \frac{\partial {g}_{i} ( x )}{\partial {λ}^{i}} , \vert {{g}^{′}}_{i} \vert N 〉 Δp$
//Accumulate gradients, see right portion of Eqs. 3-4 (307)
${Δg}_{i} ( t ) += {f}^{i} ( {g}_{i} ( x ) ) 〈 \frac{\partial {g}_{i} ( x )}{\partial {w}^{k}} , {( {( {{g}^{′}}_{i} )}^{- 1} )}^{T} \vert {{g}^{′}}_{i} \vert N 〉 Δp$

End
//update the registration parameteres for each image (308)
gᵢ(t + Δt) = gᵢ(t) + ΔtΔgᵢ(t) //discretized Eqs. 3-4

End
//update the segmentation of the ellipse (309)
λⁱ(t + Δt) = λⁱ(t) + ΔtΔλⁱ(t) //discretized Eq. 6

For ellipsoid flows, an extension of the flows in 2D to 3D is possible by modifying the parameterization from an ellipse to an ellipsoid as follows:

$$ε ( s , p ) = R ( \begin{matrix}a cos s sin p \\ b sin s sin p \\ c cos p\end{matrix} ) + d$$

where sε[0,2π),pε[0,π),a,b,c are the radii, d is the center, and R is the 3D rotation matrix of the ellipsoid.

According to an embodiment of the present disclosure, an ellipse-segmenting method utilizes geodesic energy. If the contour C(t)=ε(t) is an ellipse with parameters λᵢ, the energy of the contour is:
E(ε)=∫₀¹Φ∥εₚ∥dp,
where Φ is a weighting function, which is usually designed to slow down the shrinkage of the contour at high image gradients.

Taking the derivative of the energy with respect to an independent time variable t yields:

$$\begin{matrix}\begin{matrix}{\int }_{0}^{1} \frac{ⅆ}{ⅆ t} [ Φ ‖ {ε}_{p} ‖ ] ⅆ p = {\int }_{0}^{1} {Φ}_{t} ‖ {ε}_{p} ‖ ⅆ p + {\int }_{0}^{1} Φ \frac{ⅆ}{ⅆ t} \sqrt{〈 {ε}_{p} , {ε}_{p} 〉} ⅆ p \\ = {\int }_{0}^{1} {Φ}_{t} ‖ {ε}_{p} ‖ ⅆ p + {\int }_{0}^{1} Φ 〈 {ε}_{p t} , \underbrace{{ε}_{P} / ‖ {ε}_{P} ‖}_{T} 〉 ⅆ p \\ = {\int }_{0}^{1} {Φ}_{t} ‖ {ε}_{p} ‖ ⅆ p - {\int }_{0}^{1} Φ 〈 {ε}_{t} , {T}_{p} 〉 ⅆ p\end{matrix} & \\ \frac{\partial {λ}_{i}}{\partial t} = {\int }_{0}^{1} 〈 ∇ Φ , \frac{\partial ε}{\partial {λ}^{i}} 〉 ‖ {ε}_{p} ‖ ⅆ p - {\int }_{0}^{1} Φ 〈 \frac{\partial ε}{\partial {λ}^{i}} , {T}_{p} 〉 ⅆ p & ( 12 )\end{matrix}$$

where the T(p) is the ellipse tangent vector and Tₚ its derivative (which is then in the normal direction of the ellipse) along the ellipse, and the last line is the geodesic evolution equation for the ellipse parameters λᵢ.

Taking a closer look at this flow, one can note the similarity to the geodesic contour flow, where the second term is the curvature term weighted by the conformal factor Φ, and the first term is the gradient of the conformal factor that pulls the contour back to the real boundary. In the above equation though, the integration around the ellipse provides a significant increase in robustness of the flow, allowing the contour to escape from local minima more easily, in contrast to a generic contour with geodesic energy.

A separate ellipse is utilized for geodesic joint segmentation and registration. It is assumed that a common contour/ellipse E lives on an independent domain, and let ε¹=g₁(ε), ε²=g₂(ε) (for simplicity in illustration, m=2 ellipses).

$$\begin{matrix}E ( ε , {g}_{1} , {g}_{2} ) = {\int }_{{ε}^{1}} {Φ}^{1} ( {x}_{1} ) ‖ {{ε}^{1}}_{p} ‖ ⅆ p + {\int }_{{ε}^{2}} {Φ}^{2} ( {x}_{2} ) ‖ {ε}_{p}^{2} ‖ ⅆ p \\ = {\int }_{0}^{1} {Φ}^{1} ( {g}_{1} ( x ) ) ‖ {g}_{1}^{′} ( {ε}_{p} ) ‖ ⅆ p + \\ {\int }_{0}^{1} {Φ}^{2} ( {g}_{2} ( x ) ) ‖ {g}_{2}^{′} ( {ε}_{p} ) ‖ ⅆ p\end{matrix} \qquad ( 13 )$$

The evolution of the ellipse E is given by:

$$\begin{matrix}\frac{\partial E}{\partial t} = {\int }_{0}^{1} \frac{ⅆ}{ⅆ t} [ {Φ}^{1} ( {g}_{1} ( x ) ) ‖ {ε}_{p}^{1} ‖ ] ⅆ p + {\int }_{0}^{1} \frac{ⅆ}{ⅆ t} [ {Φ}^{2} ( {g}_{2} ( x ) ) ‖ {ε}_{p}^{2} ‖ ] ⅆ p \\ = {\int }_{0}^{1} {Φ}_{t}^{1} ( {g}_{1} ( x ) ) ‖ {g}_{1}^{′} ( {ε}_{p} ) ‖ ⅆ p + {\int }_{0}^{1} {Φ}_{t}^{2} ( {g}_{2} ( x ) ) ‖ {g}_{2}^{′} ( {ε}_{p} ) ‖ ⅆ p + \\ {Φ}^{1} ( {g}_{1} ( x ) ) \frac{ⅆ}{ⅆ t} \sqrt{〈 {g}_{1}^{′} ( {ε}_{p} ) , {g}_{1}^{′} ( {ε}_{p} ) 〉} ) ⅆ p + \\ {Φ}^{2} ( {g}_{2} ( x ) ) \frac{ⅆ}{ⅆ t} \sqrt{〈 {g}_{2}^{′} ( {ε}_{p} ) , {g}_{2}^{′} ( {ε}_{p} ) 〉} ) ⅆ p \\ = {\int }_{0}^{1} {Φ}_{t}^{1} ( {g}_{1} ( x ) ) ‖ {g}_{1}^{′} ( {ε}_{p} ) ‖ + {Φ}_{t}^{2} ( {g}_{2} ( x ) ) ‖ {g}_{2}^{′} ( {ε}_{p} ) ‖ ⅆ p - \\ {\int }_{0}^{1} {Φ}^{1} ( {g}_{1} ( x ) ) 〈 {( {g}_{1}^{′} ( {ε}_{p} ) )}_{t} , \underbrace{{g}_{1}^{′} ( {ε}_{p} ) / ‖ {g}_{1}^{′} ( {ε}_{p} ) ‖}_{{T}^{1}} 〉 ⅆ p - \\ {\int }_{0}^{1} {Φ}^{2} ( {g}_{2} ( x ) ) 〈 {( {g}_{2}^{′} ( {ε}_{p} ) )}_{t} , \underbrace{{g}_{2}^{′} ( {ε}_{p} ) / ‖ {g}_{2}^{′} ( {ε}_{p} ) ‖}_{{T}^{2}} 〉 ⅆ p \\ = {\int }_{0}^{1} {Φ}_{t}^{1} ( {g}_{1} ( x ) ) ‖ {g}_{1}^{′} ( {ε}_{p} ) ‖ - {Φ}^{1} ( {g}_{1} ( x ) ) 〈 {( {g}_{1}^{′} ( ε ) )}_{t} , {T}_{p}^{1} 〉 ⅆ p + \\ {\int }_{0}^{1} {Φ}_{t}^{2} ( {g}_{2} ( x ) ) ‖ {g}_{2}^{′} ( {ε}_{p} ) ‖ - {Φ}^{2} ( {g}_{2} ( x ) ) 〈 {( {g}_{2}^{′} ( ε ) )}_{t} , {T}_{p}^{2} 〉 ⅆ p\end{matrix}$$

Therefore, the evolution of the parameters of the ellipse λᵢ are given by:

$$\begin{matrix}\frac{\partial {λ}_{i}}{\partial t} = {\int }_{0}^{1} 〈 ∇ {Φ}^{1} ( {g}_{1} ( x ) ) , \frac{\partial {g}_{1} ( ε )}{\partial {λ}^{i}} 〉 ‖ {g}_{1}^{′} ( {ε}_{p} ) ‖ - \\ {\int }_{0}^{1} {Φ}^{1} ( {g}_{1} ( x ) ) 〈 \frac{\partial {g}_{1} ( ε )}{\partial {λ}^{i}} , {T}_{p}^{1} 〉 ⅆ p + \\ {\int }_{0}^{1} 〈 ∇ {Φ}^{2} ( {g}_{2} ( x ) ) , \frac{\partial {g}_{2} ( ε )}{\partial {λ}^{i}} 〉 ‖ {g}_{2}^{′} ( {ε}_{p} ) ‖ - \\ {\int }_{0}^{1} {Φ}^{2} ( {g}_{2} ( x ) ) 〈 \frac{\partial {g}_{2} ( ε )}{\partial {λ}^{i}} , {T}_{p}^{2} 〉 ⅆ p\end{matrix} \qquad ( 14 )$$

The curvature-like term, i.e. the term multiplied by the conformal factor Φⁱ, is not of much use for an ellipse, since it is always convex. Therefore, such terms are eliminated. The influence of the gradient of Φⁱ is extended by utilizing a gradient vector flow method to prevent the flow in Eq. (14) from staying too local.

A geodesic ellipse registration evolution is employed. The energy for the first registration g, is given by:
E(g₁)=∫₀¹Φ¹(x₁)∥εₚ¹∥dp=∫₀¹Φ¹(g₁(x))∥g₁′(εₚ)∥dp (15)

Then the evolution of the first registration g₁ can be obtained as follows:

$$\frac{\partial {( {g}_{1} )}_{i}}{\partial t} = \frac{\partial E}{\partial {( {g}_{1} )}_{i}} = {\int }_{0}^{1} 〈 ∇ {Φ}^{1} ( {g}_{1} ( x ) ) , \frac{\partial {g}_{1} ( ε )}{\partial {w}_{i}} 〉 ‖ {g}_{1}^{′} ( {ε}_{p} ) ‖ ⅆ p , \qquad ( 16 )$$

and similarly the evolution of the second registration g₂

$$\frac{\partial {( {g}_{2} )}_{i}}{\partial t} = \frac{\partial E}{\partial {( {g}_{2} )}_{i}} = {\int }_{0}^{1} 〈 ∇ {Φ}^{2} ( {g}_{2} ( x ) ) , \frac{\partial {g}_{2} ( ε )}{\partial {w}_{i}} 〉 ‖ {g}_{2}^{′} ( {ε}_{p} ) ‖ ⅆ p . \qquad ( 17 )$$

The combined region and boundary based ellipse flow utilizes both the region and boundary information in segmentation and registration of structures in medical images. These images have a high variability in the condition of the border and intensity information of the target structure. A weighted combination of the region and geodesic flows are used to obtain the resulting update equations for both the registration and the segmentation of the ellipse as follows:

$$\begin{matrix}\begin{matrix}\frac{\partial {( {g}_{k} )}_{i}}{\partial t} = \frac{\partial E}{\partial {( {g}_{k} )}_{i}} \\ = {\int }_{0}^{1} 〈 [ α ∇ {Φ}^{k} ( {g}_{k} ( x ) ) + ( 1 - α ) {f}^{k} ( {g}_{k} ( x ) ) N ] , \frac{\partial {g}_{k} ( x )}{\partial {w}_{i}} 〉 \\ ‖ {g}_{k}^{′} ( {ε}_{p} ) ‖ ⅆ p ,\end{matrix} & ( 18 ) \\ \begin{matrix}\frac{\partial {λ}_{i}}{\partial t} = {\int }_{0}^{1} 〈 \frac{\partial {g}_{1} ( ε )}{\partial {λ}_{i}} , [ α ∇ {Φ}^{1} ( {g}_{1} ( x ) ) + ( 1 - α ) {f}^{1} ( {g}_{1} ( x ) ) N ] 〉 \\ ‖ {g}_{1}^{′} ( {ε}_{p} ) ‖ ⅆ p + \\ {\int }_{0}^{1} 〈 \frac{\partial {g}_{2} ( ε )}{\partial {λ}_{i}} , [ α ∇ {Φ}^{2} ( {g}_{2} ( x ) ) + ( 1 - α ) {f}^{2} ( {g}_{2} ( x ) ) N ] 〉 \\ ‖ {g}_{2}^{′} ( {ε}_{p} ) ‖ ⅆ p\end{matrix} & ( 19 )\end{matrix}$$

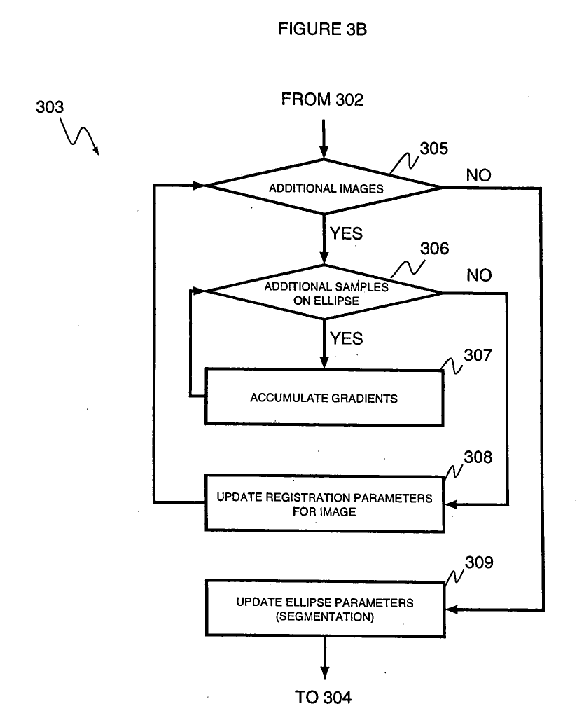

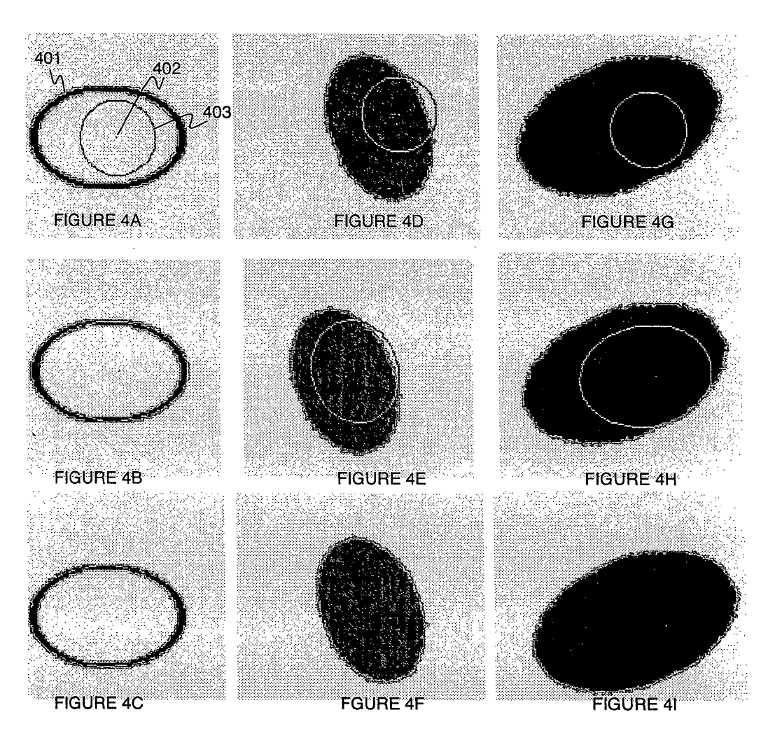

for the ith parameter of both the registration and segmentation i=1, . . . , 5, and for the kth transformation to image k, (k=1,2 here for simplicity). The flows in Eqs. (18-19) were tested on a set of three synthetic objects, each subject to different rotation, translation and scaling. FIGS. 4A, 4D and 4G show an initialization of ellipses, e.g., 402 and 403. FIGS. 4B, 4E and 4H shown a respective interim step. FIGS. 4C, 4F and 4I show a respective converged result. Each of FIGS. 4A-4I depicts a synthetic object to be detected, e.g., 401, a seed position 402 and an ellipse, e.g., 403. An equal weighting from both geodesic and region-based terms is used, where α=0.5. Note that these examples also demonstrates that an initialization that is only good for some images still leads to a stable result on all images.

General Contour with Non-Rigid Registration

The non-rigid deformation corresponds to a registration in infinite dimensions without using shape priors. If required by the application though, the shape priors can be incorporated into the framework. The joint segmentation and registration accounts for general problems of registration among anatomical structures defined by a deformation field or a displacement field between target regions.

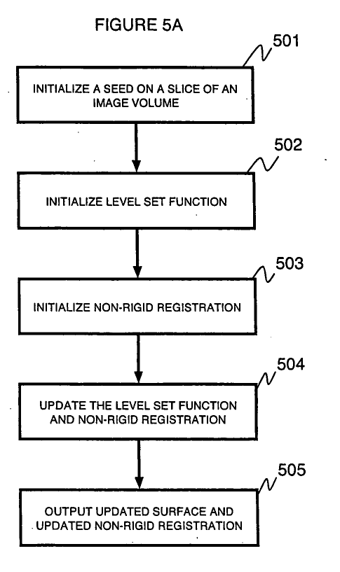

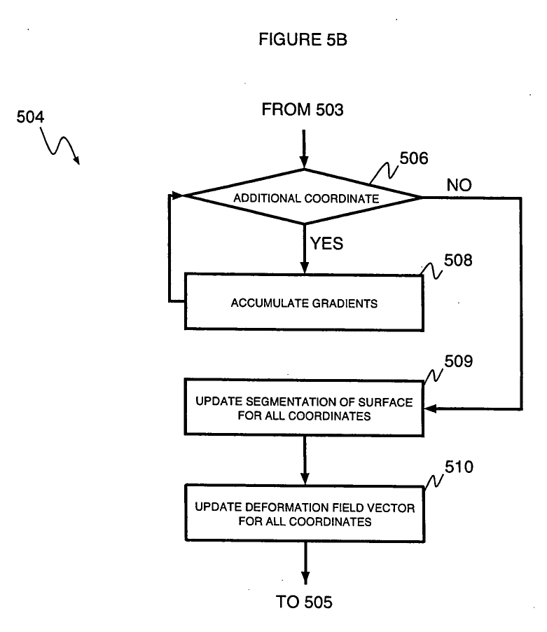

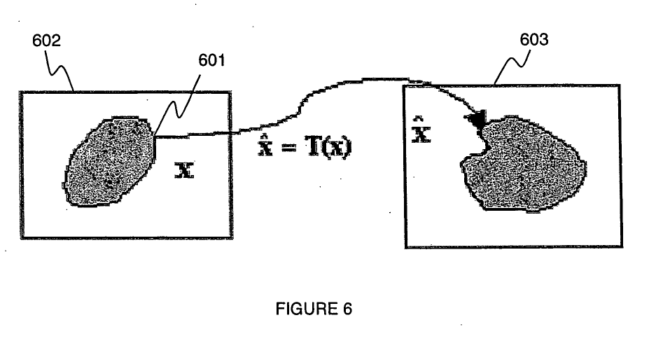

Referring to FIG. 5A, a joint non-rigid registration and segmentation method includes initializing a seed on a slice of an image volume 501, for example, manually selecting the seed. Based on the initialized seed, a level set function is initialized 502 that is a signed distance function, negative inside and positive outside the surface, which is converted into a 3D surface seed by extending to two neighboring slices. A non-rigid registration is initialized 503, which is a deformation vector field to a zero vector field over the image domain. The surface's level set function and deformation vector field are updated 504, and the updated surface, either its signed distance function over the domain or the surface that is the zero-level set of the signed distance function, and the updated deformation field U(TotalTime), which maps the surface in one of the images onto another image, are output 505. FIG. 6 illustrates a point 601 in a first domain 602 being transformed or mapped into a second domain 603.

Given two image volumes, the transformation that deforms one of the images to the other one may be denoted by: \hat{x}=T(x)=x+u(x), where u(x) is the displacement vector field defined as u:Ω→ [symbol] ⁿ (n=2 or 3).

The general region-based energy functional for a joint segmentation and registration purpose can be rewritten as follows:
E(S,u)=∫_{S}_{ᵢₙ}fᵢₙ(x)dx+∫_{S}_{ₒᵤₜ}fₒᵤₜ(x)dx+∫_{S}dA∫_{Ŝ}_{ᵢₙ}\hat{f}ᵢₙ(\hat{x})d\hat{x}+∫_{Ŝ}_{ₒᵤₜ}\hat{f}ₒᵤₜ(\hat{x})d\hat{x}+∫_{Ω}∥Du∥²dx (20)
where the last two terms represent the regularization on the unknown surface S and vector field u,\hat{x}=x+u(x), and fᵢₙ, fₒᵤₜ, \hat{f}ᵢₙ and \hat{f}ₒᵤₜ are as before.

The solutions to the minimization problems may be given by:

$$\tilde{S} = arg {min}_{S} E ( S , u ) , and \tilde{u} = arg {min}_{u} E ( S , u ) \qquad ( 21 )$$

Let us reformulate the problem in terms of a level set function Φ:Ω→ [symbol] that represents S as its zero level set. Let H denote a Heaviside function, then E in Eq. (20) can be re-written as:
E(Φ,u)=∫_{Ω}fᵢₙ(x)H(Φ(x))dx+∫_{Ω}fₒᵤₜ(x)(1−H(Φ(x)))dx+∫_{\hat{Ω}}\hat{f}ᵢₙ(\hat{x})H(\hat{Φ}(\hat{x}))d\hat{x}+∫_{\hat{Ω}}\hat{f}ₒᵤₜ(\hat{x})(1−H(\hat{Φ}(\hat{x})))d\hat{x}+∫_{Ω}δ(Φ)|∇Φ|dx+∫_{Ω}∥Du(x)∥²dx (21)
where \hat{Φ}(x+u)=Φ(x) and δ(x)=dH(x)/dx in the sense of distributions. Regularized versions of H(x) and δ(x) may be used, for example, according a Heaviside function and delta function, respectively. The surface evolution in this case is given by:

$$\frac{\partial Φ}{\partial t} = δ ( Φ ) f ( x ) + δ ( \hat{Φ} ( x + u ( x ) ) ) \hat{f} ( x + u ( x ) ) + κ . \qquad ( 22 )$$

For registration evolution, the only part of the energy functional in Eq. (21) that is taken into account is:

$$E ( u ) = {\int }_{Ω} \underbrace{{\hat{f}}_{i n} ( x + u ( x ) ) H ( \hat{Φ} ( x + u ( x ) ) )}_{{F}_{i n} ( x + u ( x ) )} ⅆ x + {\int }_{Ω} \underbrace{{\hat{f}}_{out} ( x + u ( x ) ) ( 1 - H ( \hat{Φ} ( x + u ( x ) ) ) )}_{{F}_{out} ( x + u ( x ) )} ⅆ x + α {\int }_{Ω} {‖ Du ‖}^{2} ⅆ x \qquad ( 23 )$$

The gradient of E with respect to u is

$$\frac{\partial E}{\partial u} = - ∇ u {F}_{in} ( u ) - ∇ u {F}_{out} ( u ) + α Δ u ,$$

and we solve it by a gradient descent PDE (over domain Ω) whose steady state solution gives the minimizer displacement field u that varies over space:

$$\begin{matrix}\frac{\partial u ( x , t )}{\partial t} = ∇ u {\hat{f}}_{i n} ( x + u ) H ( \hat{Φ} ( x + u ( x ) ) ) + ∇ u {\hat{f}}_{out} ( x + u ) ( 1 - H ( \hat{Φ} ( x + u ( x ) ) ) ) + \hat{f} ( x + u ) δ ( \hat{Φ} ( x + u ( x ) ) ) ∇ \hat{Φ} ( x + u ) + αΔ u ( x ) & \\ u ( x , 0 ) = {u}_{o} ( x ) & ( 24 )\end{matrix}$$

Due to a narrowband level set implementation, the method is effectively solving for u(x) on a band around the surface.

Referring to FIG. 5B, the following method may be used for updating the surface's level set function and the deformation vector field.

ΔU(X,t) = 0, for all X in the image domain,
ΔΦ(t) = 0, for all X in the image domain
where X is a coordinate, e.g., (x,y) for 2D or (x,y,z) for 3D
For X in the image volume domain (506)

//Accumulate gradients for surface update (508)
ΔΦ(X,t)+= right portion of Eq.22
//Accumulate gradients for deformation field update (508)
ΔU(X,t)+= right portion of Eq.24

End
For X in the image volume domain

//Update the segmentation of the surface (509)
Φ(X,t + Δt) = Φ(X,t) + ΔtΔΦ(X,t)
//Update the deformation field (510)
U(X,t + Δt) = U(X,t) + ΔtΔU(X,t) //Discretized Eq.24

End

If as a special case, a piecewise (p-w) constant model known as “Chan-Vese” type flows is chosen for the target regions that are to be segmented and registered in images I and Î, the region based term in the energy, and the gradient terms can be given by:

$$\begin{matrix}\hat{f} = {\hat{f}}_{i n} - {\hat{f}}_{out} \\ = 2 ( {\hat{m}}_{i n} - {\hat{m}}_{out} ) ( \frac{{\hat{m}}_{i n} + {\hat{m}}_{out}}{2} - \hat{I} ( x + u ( x ) ) ) ,\end{matrix}$$

$$∇ u {\hat{f}}_{i n} = 2 ( \hat{I} ( x + u ) - {\hat{m}}_{i n} ) ∇ \hat{I} ( x + u ( x ) )$$

$$∇ u {\hat{f}}_{out} = 2 ( \hat{I} ( x + u ) - {\hat{m}}_{out} ) ∇ \hat{I} ( x + u ( x ) ) .$$

These expressions can be inserted into Eq. (24) to obtain the PDE for evolution of non-rigid registration field for the p-w constant region model:

$$\begin{matrix}\frac{\partial u ( x , t )}{\partial t} = - ( {\hat{m}}_{i n} - {\hat{m}}_{out} ) [ \frac{( {\hat{m}}_{i n} + {\hat{m}}_{out} )}{2} - \hat{I} ( x + u ) ] δ ( \hat{Φ} ( x + u ( x ) ) ) ∇ \hat{Φ} ( x + u ) - ( \hat{I} ( x + u ) - {\hat{m}}_{i n} ) ∇ \hat{I} ( x + u ( x ) ) H ( \hat{Φ} ( x + u ( x ) ) ) - ( \hat{I} ( x + u ) - {\hat{m}}_{out} ) ∇ \hat{I} ( x + u ( x ) ) ( 1 - H ( \hat{Φ} ( x + u ( x ) ) ) ) + \frac{α}{2} Δ u ( x ) & \\ u ( x , 0 ) = {u}_{o} ( x ) = 0 , & ( 25 )\end{matrix}$$

where a zero vector field initialization is adequate in solving the PDE without any prior knowledge of the true vector field u.

In some segmentation applications, the basic approach of thresholding has proven to be useful. Instead of using means inside and outside the surface, one can convert such a basic “Chan-Vese” flow to a “thresholding” flow. This is also equivalent to region growing to separate the intensity inside the growing surface from the outside by the given threshold. For this purpose the following speed function may be used:
\hat{f}=(\hat{m}ᵢₙ−\hat{m}ₒᵤₜ)(T−Î(x+u(x))) (26)
where

$$\frac{{\hat{m}}_{i n} + {\hat{m}}_{out}}{2}$$

quantity in “Chan-Vese” flow is replaced by an arbitrary threshold T. For this speed function the following PDE may be used for updating the vector field:

$$\begin{matrix}\begin{matrix}\frac{\partial u ( x , t )}{\partial t} = - ( {\hat{m}}_{i n} - {\hat{m}}_{out} ) ( T - \hat{I} ( x + u ) ) \\ δ ( \hat{Φ} ( x + u ( x ) ) ) ∇ \hat{Φ} ( x + u ) + \frac{α}{2} Δ u ( x )\end{matrix} & \\ u ( x , 0 ) = {u}_{o} ( x ) = 0 , & ( 27 )\end{matrix}$$

with the boundary term only.

Having described embodiments for a system and method for a joint segmentation and registration of images for object detection, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

What is claimed is:

1. A method for joint segmentation and registration of a contour for detecting an object in image data comprising:
initializing a segmentation;
initializing a registration of the segmentation;
updating the segmentation and the registration; and
outputting an updated segmentation and an updated registration, wherein the updated segmentation and updated registration delineates the object in the image data.

2. The method of claim 1, wherein the segmentation is an ellipse and the registration is rigid.

3. The method of claim 2, wherein updating the segmentation and registration further comprises:
accumulating gradients for the segmentation evolution for a plurality of images in the image data and a plurality of samples on the ellipse;
updating the registration for each of the plurality of images; and
updating the segmentation given the updated registration.

4. The method of claim 2, further comprising determining parameters of each rigid registration including a translation vector, a rotation matrix and a non-uniform scale matrix.

5. The method of claim 1, wherein the segmentation is a surface of a three-dimensional structure and the registration is non-rigid.

6. The method of claim 5, wherein the non-rigid registration is a deformation vector field over the surface of the three-dimensional object.

7. The method of claim 6, wherein updating the segmentation and registration further comprises:
accumulating gradients for surface evolution for each coordinate in the image volume; and
accumulating gradients for the non-rigid registration evolution for each coordinate in the image volume.

8. The method claim 6, wherein the deformation vector field maps the surface in a first image onto the surface of in a second image.

9. The method of claim 5, wherein the three-dimensional surface is determined from an initialized surface seed of the segmentation in a first image slice of the image data and two neighboring slices in the image data in which the surface seed is extended.

10. A program storage device readable by machine, tangibly embodying a program of instructions executable by the machine to perform method steps for joint segmentation and registration of a contour for detecting an object in image data, the method steps comprising:
initializing a segmentation;
initializing a registration of the segmentation;
updating the segmentation and the registration; and
outputting an updated segmentation and an updated registration, wherein the updated segmentation and updated registration delineates the object in the image data.

11. The method of claim 10, wherein the segmentation is an ellipse and the registration is rigid.

12. The method of claim 11, wherein updating the segmentation and registration further comprises:
accumulating gradients for the segmentation evolution for a plurality of images in the image data and a plurality of samples on the ellipse;
updating the registration for each of the plurality of images; and
updating the segmentation given the updated registration.

13. The method of claim 10, wherein the segmentation is a surface of a three-dimensional structure and the registration is non-rigid.

14. The method of claim 13, wherein the non-rigid registration is a deformation vector field over the surface of the three-dimensional object.

15. The method of claim 14, wherein updating the segmentation and registration further comprises:
accumulating gradients for surface evolution for each coordinate in the image volume; and
accumulating gradients for the non-rigid registration evolution for each coordinate in the image volume.

16. A method for joint segmentation and registration of a contour for detecting an object in an image volume comprising:
initializing a seed in a first slice of the image volume;
initializing a signed distance function;
converting the distance function into a three-dimensional surface seed;
initializing a non-rigid registration for the surface;
updating the distance function and the non-rigid registration; and
outputting an updated surface and distance function mapping the surface in the first slice to the surface in a second slice, the surface delineating the object in the image volume.

17. The method of claim 16, wherein updating the segmentation and registration further comprises:
accumulating gradients for surface evolution for each coordinate in the image volume; and
accumulating gradients for the non-rigid registration evolution for each coordinate in the image volume.

18. The method of claim 16, wherein the surface is determined from an initialized surface seed of the segmentation in the first image slice of the image data and two neighboring slices in the image data in which the surface seed is extended.
